(19) United States
(12) Patent Application Publication
(10) Pub. No.: US 2002/0110834 A1
(43) Pub. Date: Aug. 15, 2002

(54) Fluorescent assay for proteolysis

(76) Inventors: Stephen J. Benkovic, State College, PA (US); Charles P. Scott, Narberth, PA (US)

Correspondence Address: MCKEE, VOORHEES & SEASE, P.L.C., ATTN: PENNSYLVANIA STATE UNIVERSITY, 801 GRAND AVENUE, SUITE 3200, DES MOINES, IA, 50309-2721, US

(21) Appl. No.: 10/071,468

(22) Filed: Feb. 8, 2002

Related U.S. Application Data
(63) Continuation of application No. 09/713,614, filed on Nov. 15, 2000, now Pat. No. 6,346,924.
(63) Continuation of application No. 08/817,445, filed on Apr. 30, 1997, now Pat. No. 6,198,458.
(60) Provisional application No. 60/267,440, filed on Feb. 8, 2001.

(30) Foreign Application Priority Data
Nov. 4, 1994 (NZ) 264864
Aug. 15, 1995 (NZ) 272778
Oct. 16, 1995 (NZ) PCT/NZ95/00106

(51) Int. Cl. G01N 33/53

(52) U.S. Cl. 435/7.1

(57) ABSTRACT

The invention includes methods for assaying protease activity. According to one aspect of the present invention provides a nucleic acid construct having a sequence encoding an amino terminal portion of a fluorescent reporter fused to a sequence encoding a substrate of a protease followed by a sequence encoding a carboxyl terminal portion of a fluorescent reporter protein. The recombinant fluorescent substrate is then expressed in the presence of a protease. A change in quenching of fluorescence in the recombinant substrate is then detected. The change is an indication of protease activity.

### PRIORITY CLAIM

This application claims priority to Provisional Application Serial No. 60/267,440, filed on Feb. 8, 2001, herein incorporated by reference in its entirety.

### GRANT REFERENCE

Work for this invention was funded in part by a grant from the National Institute of Health, Grant No. GM19891. The government may have certain rights in this invention.

### FIELD OF INVENTION

This invention relates generally to the field of biotechnology. More particularly, the present invention relates to a fluorescent assay for proteolysis.

### BACKGROUND OF THE INVENTION

Proteolysis is a fundamental regulatory mechanism. For example, proteolysis is responsible for the activating and maturing polypeptides, degrading misfolded and damaged proteins, and the controlled turnover of peptides within a cell. Of course, one skilled in the art will recognize that proteolysis is a key mechanism in a number of biological systems. Therefore, understanding and control of proteolysis is potentially applicable to vast numbers of applications, including, but not limited to pharmaceutical development, curing diseases, and other biochemical applications. One of the primary challenges presented by proteolysis is identifying specific proteases or specific protease inhibitors.

One prior art approach involves fluorescence resonance energy transfer (FRET). See Mahajan, N. P., Harrison-Shostak, D. C., Michaux, J. and Herman, B., Chem. Biol., 1999, 6, 401-09, herein incorporated by reference in its entirety. In the FRET approach, an amino-acid sequence is introduced as a linker between different fluorescent proteins. For example, the amino-acid sequences are introduced between blue fluorescent protein (BFP) and green fluorescent protein (GFP) or between cyan fluorescent protein and yellow fluorescent protein. These fluorescent substrates are cleaved by a particular protease. FRET is used to determine the proximity between the fluorophores. When the link is present, excitation energy applied to the first fluorophores is transferred to the second fluorophore, the second fluorophore will fluoresce at an emission maximum. When cleaved by the protease, the distance between the fluorophores increases and there is a loss in FRET and therefore fluorescence. Although such an approach could be used for highthroughput screening, problems remain.

Another prior art approach is the intracellular protease assay described by Sice, H. J. and Kristie, T. M., Proc. Natl. Acad. Sci. USA, 1998, 95, 2828 (herein incorporated by reference in its entirety). In this approach, a phage-encoded repressor containing a site-specific protease is expressed. The repressor prohibits expression of the phage lytic replication functions. If the site-specific protease is not appropriate, there is no effect on the recombinant repressor. When the appropriate site-specific protease is encoded, the protease cleaves the recombinant repressor, resulting in lytic replication.

One significant limitation on this approach is that the assay is indirect. Proteolysis does not lead directly to the change in signal. Rather, proteolysis leads to the loss of the activity of a protein (i.e. the protein substrate is a transcriptional repressor) which then subsequently and indirectly leads to a signal (i.e. transcription of a reporter gene). A direct signal generation would be preferable. Only direct signal systems are amenable to being used as extracellular assays. In the instant application, Applicants have used a fluorescent protein, particularly green fluorescent protein (GFP), as both an intracellular and extracellular detection marker wherein the GFP quenching upon expression of a protease reflects proteolysis of the GFP substrate molecule rather than suppression of GFP expression. It has been shown that the amino- and carboxyl-terminal fragments of GFP fail to associate without facilitation. The peptide bond between the amino and carboxyl-terminal fragment must therefore be essential to generate or maintain fluorescence. (See WO 01/87919 A2 to Regan et al., herein incorporated by reference in its entirety). Thus, while Regan et al. discloses a strategy for the noncovalent reconnection of the N- and C-termini of a dissected surface loop of a protein by means of antiparallel leucine zippers, Applicants, however, show that the insertion of a protease susceptible site into a surface exposed loop of a intrinsically fluorescent protein, such as GFP, will convert it into a intracellular substrate for a protease, resulting in protease-dependent quenching of GFP fluorescence.

Another variation on the direct assay is to have proteolysis either cause or prevent cell death. See Grafstrom, R. H., Zachariasewcyz, K., Brigandi, R. A., Block, T. M., Adv. Exp. Med. Biol., 1992, 312, 25-40 (herein incorporated by reference in its entirety). This approach provides a selection that is a very powerful way to screen large libraries of inhibitors. Nevertheless, problems remain. In particular, cell death is the reporter, so there may not be a way to use the substrate as an assay outside of a cell. A further problem is that selection creates an evolutionary system wherein cells that mutate the protease have a survival advantage. This can be a very significant problem when screening inhibitor libraries. Finally, such an assay can only be used in one direction: survival

Another approach involves using a screen in which the substrate of the protease is itself an enzyme (particularly, β-galactosidase). See Baum, E. Z., Bebernitz, G. A., Gluzman, Y., Proc. Natl. Acad. Sci. USA, 1990, 87, 10023-10027. According to this approach, activity of the protease is detected by loss of activity of a second (coupling) enzyme. Assaying a coupled enzyme generally, if not always, requires the addition of a substrate (X-gal for β-galactosidase, luciferin for luciferases). Requirement of the addition of a substrate is therefore one disadvantage of this approach. Furthermore the colormetric (β-gal) and luminescence (Luciferase)-based screens of this approach provide only for a limited throughput screening process.

U.S. Pat. No. 6,180,343 to Anderson et al., herein incorporated by reference in its entirety, describe constructs in which a peptide or library of peptides is introduced in an internal position within GFP to find peptides that modulate cell phenotype. Anderson et al. Use the GFP portion of the fusion construct to report on peptide expression level rather than as an element of a phenotypic screen. There is no language in the patent or claims describing the GFP-peptide fusion product as a substrate that would report on the activity of an enzyme via changes in fluorescence emission. In particular, protease-induced quenching of GFP fluorescence is unlikely to have envisioned by the authors since U.S. Pat. No. 6,180,343 predates literature reports of the poor complementarity of amino and carboxyl-terminal fragments of GFP (Ghosh et al. JACS 2000) by almost two years.

Therefore, it is a primary object of the present invention to improve upon the state of the art of protease screening using fluorescence or changes in fluorescence as a reporter.

Another object, feature, or advantage of the present invention is to provide a method for assaying protease activity that can be used to identify proteases that cleave a target amino acid sequence.

A further object, feature, or advantage of the present invention is to provide a method for determining a substrate recognized by a test protease.

Yet another object, feature, or advantage of the present invention is to provide a method that provides for direct signaling of proteolytic activity.

Another object, feature, or advantage of the present invention is to provide an assay for proteolysis that does not require additional components.

A still further object, feature, or advantage of the present invention to provide for an assay for proteolysis that is capable of being used for high throughput screening.

It is a still further object feature, or advantage of the present invention to provide for an assay for proteolysis that it is compatible with both intracellular and extracellular applications.

### SUMMARY OF THE INVENTION

The present invention is a fluorescent assay for proteolysis. The present invention is used in a number of different applications. For example, the present invention is used to identify or screen proteases that cleave a particular protease substrate. Also, the present invention can be used to identify or screen protease substrates that are cleaved by a particular protease. Proteases have numerous biotechnology applications, including but not limited to uses as a major target for drug action and development.

Accordingly, it is valuable to the field of pharmaceutical development to identify and characterize previously unknown protease nucleic acids and polypeptides.

The present invention relies upon the presence of a peptide bond between the amino and carboxyl terminal fragment of a fluorescent substrate being essential to generate or maintain fluorescence. With this in mind, the present invention uses a construct having a protease substrate fused between an amino terminal portion of a fluorescent reporter protein and a carboxyl-terminal portion of the fluorescent reporter protein. The protease substrate is then expressed in the presence of the protease. Changes in the quenching of fluorescence in the recombinant substrate are then observed. The changes in the quenching of fluorescence in the recombinant substrate directly signal protease activity. Where fluorescence is quenched, proteolytic activity has cleaved the fluorescent reporter protein into fragments with little intrinsic affinity for one another.

### BRIEF DESCRIPTION OF THE DRAWINGS

FIG. 1 provides a pictorial representation of proteolysis cleaving GFP into two fragments.

FIG. 2 provides flow cytometry analysis of a construct expressing the NS3/4A protease from HCV and a mutant of the active site serine (S139G).

FIG. 3 provides a Western blot having a first lane of a wild-type GFP control, a second lane with GFP having a 20 amino acid protease substrate insert, a third lane having the GFP protease substrate co-transformed into bacterial with the protease, and a fourth lane with the GFP protease substrate co-transformed into bacteria with the protease and the protease expression induced.

FIGS. 4A and 4B provide flow cytometry and fluorescence microscopy for induced cultures for a MMP-I protease.

### DETAILED DESCRIPTION

A number of definitions of terms are contained herein. Such definitions are consistent with the use such terms in the art. Any terms used herein but otherwise not defined are used consistent with their definitions within the art.

As used herein, a “fusion protein” is a protein consisting of more than one polypeptides or parts of polypeptides that are operably linked together. Similarly, a “fusion nucleic acid” is a nucleic acid encoding a fusion protein. Furthermore, as used herein, “fuse” means operably linked.

As used herein, a “GFP” or “Green Fluorescent Protein” includes GFP derivatives or variants, including insertions, deletions, or substitutions of amino acids provided fluorescence is exhibited at approximately 490 nm to 600 nm.

As used herein, a “substrate” is generally a chemical species or biomolecule the reaction of which is under observation. A “protease substrate” includes amino acid sequences that are cleaved by a protease.

As used herein, a “fluorescent reporter” includes fluorophores that can be assayed such as, but not limited to, GFP.

As used herein, a “construct” is generally an assembly of constituent parts.

As used herein, an “amino terminal portion” refers to the portion of a peptide having a free amino group or N terminus. A “carboxyl terminal portion” refers to that portion of a peptide having a free carboxyl group or C terminus.

As used herein, “quenching” means suppressing or attenuating.

As used herein, “expressing” means the transcription and/or translation.

As used herein, “random peptide” refers to a combination of two or more amino acid residues and constructed by a means with which one does not preselect the complete sequence of a particular oligomer.

As used herein, “random peptide library” refers to a library comprising not only of a set of recombinant DNA vectors (also called recombinants) that encodes a set of random peptides, but also the fusion proteins containing those random peptides.

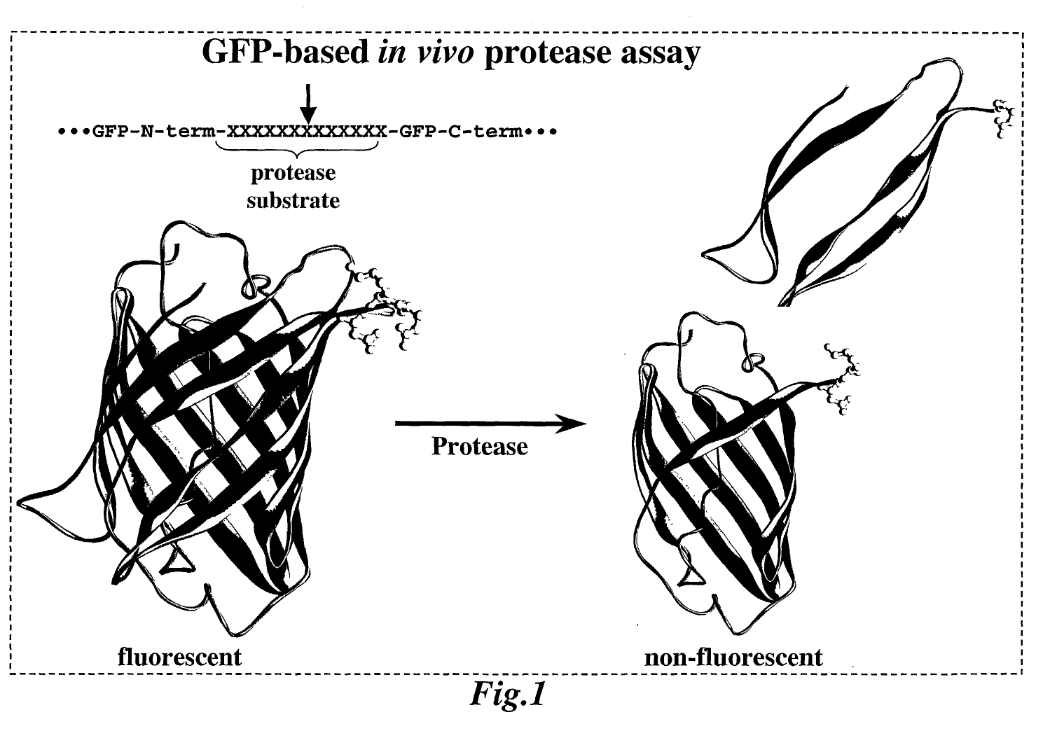

The present invention provides for screening for protease activity inside cells based on fluorescence quenching. Regan et al (J. Am. Chem. Soc., 2000, 122, 5658-9), Nagai et al (Proc. Natl. Acad. Sci. USA 2001, 98, 3197-3202) and Umezawa et al (Anal. Chem. 2000, 72, 5151-7) have all shown that amino-and carboxyl-terminal fragments of green fluorescent protein (GFP) fail to associate without facilitation. The peptide bond between the amino and carboxyl-terminal fragment must therefore be essential to generate or maintain fluorescence. The Applicants have shown that insertion of a protease-susceptible site into a surface exposed loop of an intrinsically fluorescent protein (such as GFP) converts it into an intracellular substrate for a protease. Proteolytic activity would be expected to generate two fragments with little intrinsic affinity for one another. The presence of proteolytic activity leads to protease-dependent quenching of GFP fluorescence, yielding a fluorescent assay for protolysis compatible with both intracellular and extracellular detection. This is shown in the pictorial representation of FIG. 1. In a preferred embodiment GFP is used, however the present invention contemplates that other types of fluorescent proteins can be used without undue experimentation, as other types of fluorescent proteins are known in the art and other types of fluorescent proteins are known to have similar structures to GFP.

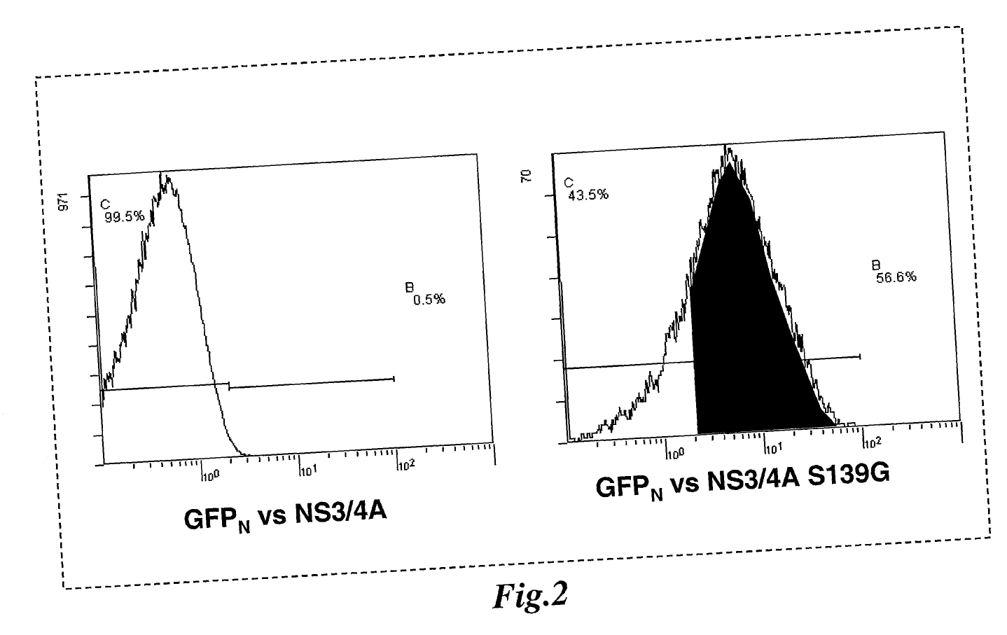

A construct expressing either the NS3/4A serine protease from HCV or a mutant NS3/4A protease in which the active site serine was converted to glycine (S139G) was co-expressed with a recombinant GFP protein having a substrate sequence for the NS3/4A protease (the NS4A/B consensus sequence) inserted between residues 157 and 158 of GFP (GFP_{N}). Bacteria were innoculated, and protease expression was induced for 24 hours at ambient temperature. The induced cultures were analyzed by flow cytometry as shown in FIG. 2. Bacteria expressing the mutant protease (right) displayed fluorescence similar to that observed with GFPN alone. However, the fluorescence of GFP_{N} in bacteria co-expressing active NS3/4A was quenched completely. Thus, the presence, absence, or attenuation, or changes in florescence is used to determine proteolytic activity.

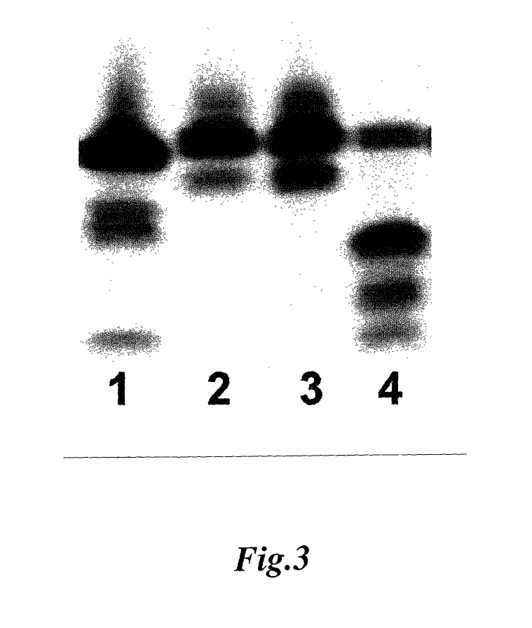

A Western blot (shown in FIG. 3) was then performed in order to verify that GFP quenching upon co-expression of the protease reflected proteolysis of the GFP substrate molecule rather than suppression of GFP expression. Lane 1 is a wild-type GFP control, lane 2 is GFP with a 20 amino acid protease substrate insert, lane 3 is the GFP protease substrate co-transformed into bacteria with the protease, but without induction of the protease, and lane 4 is the GFP protease substrate co-transformed into bacteria with the protease and with the protease expression induced. Although in this example, a 20 amino acid protease substrate is used, longer or shorter protease substrate inserts can be used. Comparison of lanes 3 and 4 indicates that induction of the protease has little or no effect on GFP expression (as judged from the sum of the intensities of bands that cross-react with a GFP-specific antibody). However, expression of the protease results in the generation of a number of low molecular weight species with concomitant loss of the full length GFP protein. Thus, the fluorescence quenching results from GFP proteolysis rather than suppression of expression of GFP upon induction of the protease, validating the mechanism of the present invention.

One skilled in the art and having the benefit of this disclosure would appreciate that the fluorescent protein based protease assay allows for high throughput screening using a fluorescence activated cell sorting (FACS) method. FACS provides the advantage of much higher throughput than (B-gal) or luminescence (Luciferase)-based screens used in the prior art.

As shown, the present invention provides for assaying for protease activity. A nucleic acid construct having a sequence encoding an amino terminal portion of a fluorescent reporter protein fused to a sequence encoding substrate of a protease followed by a sequence encoding a carboxyl terminal portion of a fluorescent reporter protein was provided. The recombinant fluorescent substrate is then expressed in the presence of the protease. The present invention provides that the protease may also, but need not, be introduced by expression from a nucleic acid construct such that there is coexpression. After expression of the recombinant fluorescent substrate, the present invention provides for detecting a change in quenching of fluorescence in the recombinant substrate as an indication of protease activity. In particular, where there is cleavage in the protease substrate sequence, fluorescence is quenched. Thus the present invention provides for direct signaling of proteolytic activity.

The present invention optionally provides for a purifying step. Such purification methods of well known to those skilled in the art. In such a variation, the present invention provides for assaying proteolytic activity between a protease and a protease substrate sequence of amino acids. The method includes inserting a nucleic acid sequence of amino acids into a surface exposed loop of an intrinsically fluorescent protein in order to form a recombinant protein. Then the recombinant protein substrate is expressed. Then the recombinant protein substrate is purified. Next, the quenching or changes in quenching of fluorescence in the presence of the protease are detected.

Another application of the present invention is in determining a substrate recognized by a test protease. In this application, each of a plurality of protease substrate sequences is inserted into a surface exposed loop of an intrinsically fluorescent protein to form a library of recombinant proteins. The library of recombinant proteins is coexpressed in the presence of a test protease. Then, members of the library of recombinant proteins having expression quenched are identified.

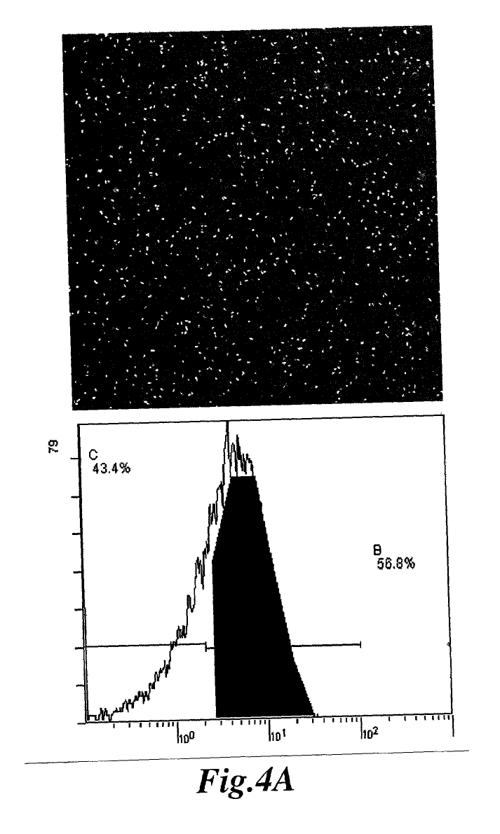

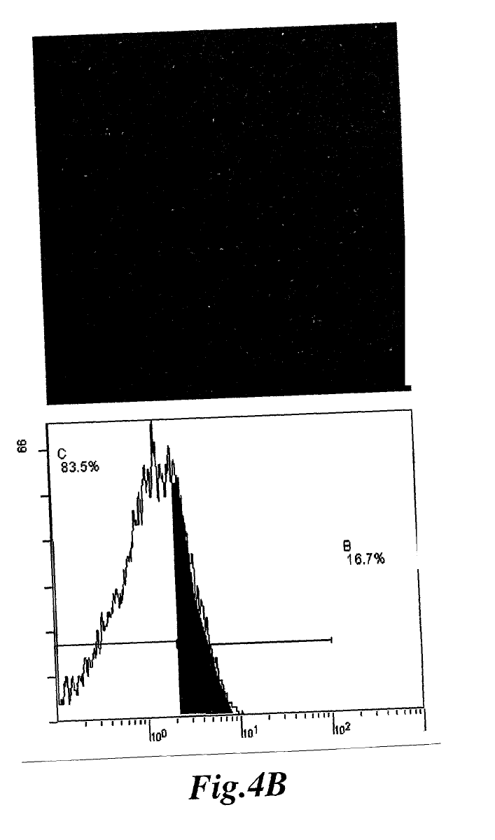

The generality of the present invention has also been demonstrated. For example, multiple proteases have been used. In one such demonstration, a matrix metallo-proteinase I(MMP-I) was used. A recombinant GFP protein having a substrate sequence for MMP-I between residues 157 and 158 of GFP (GFP_{M}) was constructed. Bacteria were inoculated, and protease expression was induced for 24 hours at ambient temperature. The induced cultures were analyzed by flow cytometry and fluorescence microscopy as shown in FIGS. 4A and 4B. Bacteria co-transformed with GFP_{M} and naked vector control (FIG. 4A displayed fluorescence similar to that observed with GFP_{M} alone. The fluorescence of GFP_{M} in bacteria co-expressing MMP-I was attenuated dramatically. Thus, one skilled in the art having the benefit of this disclosure will recognize that the methods of the present invention can be used with various proteases.

Given this flexibility of the present invention and demonstrated use of different proteases, the present invention is particularly useful for identifying a protease that cleaves a target amino acid sequence. Multiple proteases can be used in order to determine which of a plurality of proteases cleaves a target amino acid.

Those skilled in the art will recognize, or be able to ascertain, using no more than routine experimentation, many equivalents to the specific embodiments of the invention described herein. Such equivalents are intended to be encompassed by the following claims. In particular, but without limitation, the present invention contemplates variations in the fluorescent proteins used, the proteases used, the protease substrates, the size of the protease substrates, the manner of expression, and other variations.

What is claimed is:

1. A method of assaying for protease activity, comprising:
providing a nucleic acid construct having a sequence encoding an amino terminal portion of a fluorescent reporter fused to a sequence encoding a substrate of a protease followed by a sequence encoding a carboxy terminal portion of a fluorescent reporter protein;
expressing a recombinant fluorescent substrate in the presence of the protease;
detecting a change in quenching of fluorescence in the recombinant fluorescent substrate as an indication of protease activity.

2. The method of claim 1 wherein the presence of a peptide bond between the amino and carboxyl-terminal fragment of the fluorescent substrate is essential to generate or maintain fluorescence.

3. The method of claim 1 wherein fluorescence is quenched by cleavage in the protease substrate sequence.

4. The method of claim 1 wherein the intrinsically fluorescent protein is GFP.

5. The method of claim 1 wherein the protease is introduced by expression from a nucleic acid construct.

6. A method for identifying a protease that cleaves a target amino acid sequence, comprising:
providing a nucleic acid construct having a sequence encoding an amino terminal portion of a fluorescent reporter fused to a sequence encoding a desired substrate target followed by a sequence encoding a carboxy terminal portion of a fluorescent reporter protein;
expressing of the recombinant fluorescent substrate in the presence of a plurality of proteases;
detecting at least one of the plurality of proteases that recognize the target sequence by quenching of the fluorescence of the reporter.

7. The method of claim 6 wherein the fluorescent reporter protein is GFP.

8. A method for determining a substrate recognized by a test protease, comprising:
inserting each of a plurality of fusion nucleic acid sequences encoding a plurality of protease substrate sequences between sequences encoding the amino and carboxyl-terminus of an intrinsically fluorescent protein to form a library of fusion nucleic acids;
expressing the library of fusion nucleic acids to generate a library of recombinant fusion proteins in the presence of the test protease; and
identifying members of the recombinant fusion protein library having quenched fluorescence.

9. The method of claim 8 wherein the fluorescent reporter protein is GFP.

10. A method of assaying proteolytic activity between a protease and a protease substrate sequence of amino acids comprising:
(a) inserting a nucleic acid sequence of amino acids into a surface exposed loop of an intrinsically fluorescent protein to form a recombinant protein;
(b) expressing the recombinant protein substrate;
(c) purifying the recombinant protein substrate; and
(d) detecting quenching of fluorescence in the presence of a protease.

11. The method of claim 10 wherein the fluorescent protein is GFP.

12. A method of assaying proteolytic activity between a protease and a protease substrate sequence of amino acids, comprising:
(a) inserting the protease substrate sequence of amino acids into a surface exposed loop of an intrinsically fluorescent protein to form a recombinant protein;
(b) detecting quenching of fluorescence in the presence of the protease.

13. The method of claim 12 wherein the intrinsically fluorescent protein is GFP.
